(19) United States
(12) Patent Application Publication
(10) Pub. No.: US 2012/0083763 A1
(43) Pub. Date: Apr. 5, 2012

(54) Method for Delivering a Topical Agent to a Nasaogastric Passage

(76) Inventors: Edwin A. Diaz, (US); Kristianne Diaz, (US); Robert A. Diaz, The Woodlands, TX (US)

(21) Appl. No.: 12/897,656

(22) Filed: Oct. 4, 2010

(51) Int. Cl. A61M 31/00

(52) U.S. Cl. 604/514

(57) ABSTRACT

The present invention is directed to a method for delivering a topical agent to a nasogastric passage comprising: introducing a nasogastric tube into the nasogastric passage, the nasogastric tube comprising means for delivering the topical agent to the nasogastric passage; positioning the nasogastric tube such that the means for delivering the topical agent is positioned adjacent to a target area in the nasogastric passage; and delivering a therapeutic amount of the topical local anesthetic agent to the target area of the nasogastric passage.

### BACKGROUND OF THE INVENTION

1. Field of the Invention

The present invention relates to a method of delivering a topical agent to a nasal passage.

2. Background Information

Nasogastric intubation or NG insertion is a medical procedure that involves introducing a plastic tube (e.g., nasogastric tube or NG tube) into the stomach of a patient, such as a hospital patient, via the patient's nose and throat. This procedure is routinely conducted in order to feed or introduce various pharmaceutical agents into the patient. In other instances, the NG tube can be used to perform nasogastric aspiration on a patient in order to remove the contents of the patient's stomach.

While nasogastric intubation is performed on a regular basis in health care facilities around the world, one potential drawback of the procedure is that it can cause the patient pain and discomfort in areas where the NG tube is located. For instance, a patient can often experience pain and discomfort around the entrance to the nasal cavity (i.e., the nostril area of the nose). In order to reduce the amount of pain and discomfort that the patient experiences in relation to the nose, health care provides (e.g., physicians) often prescribe a topical anesthetic that can applied only around the nostrils of the patient.

While the anesthetic can alleviate the pain and discomfort that the patient experiences around the nostril area, the patient, at times, can also experience pain and discomfort in other areas of the nasaogastric passage such as the septum and/or nasal-pharynx areas that are not readily accessible.

### SUMMARY OF THE INVENTION

The present invention is directed to a method for delivering a topical agent to a nasogastric passage comprising: introducing a nasogastric tube into the nasogastric passage, the nasogastric tube comprising means for delivering the topical agent to the nasogastric passage; positioning the nasogastric tube such that the means for delivering the topical agent is positioned adjacent to a target area in the nasogastric passage; and delivering the topical agent to the target area of the nasogastric passage.

The present invention is also directed to a method for delivering a topical agent to a nasogastric passage comprising: introducing a nasogastric tube into the nasogastric passage wherein the nasogastric tube comprises an inner tube that is disposed within an outer tube, the outer tube having a diameter that is greater than the inner tube, and wherein the outer tube of the nasogastric tube further comprises a perforated portion; positioning the perforated portion of the nasogastric tube adjacent a target area in the nasogastric passage; and delivering a therapeutic amount of said topical agent to the target area of the nasogastric passage.

### DETAILED DESCRIPTION OF THE INVENTION

As used herein, unless otherwise expressly specified, all numbers such as those expressing values, ranges, amounts or percentages may be read as if prefaced by the word “about”, even if the term does not expressly appear. Plural encompasses singular and vice versa. For example, although reference is made herein to “a” topical local anesthetic, a combination (a plurality) of these components can be used in the present invention.

As used herein, “plurality” means two or more.

As used herein, “includes” and like terms means “including without limitation.”

When referring to any numerical range of values, such ranges are understood to include each and every number and/or fraction between the stated range minimum and maximum. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

### Method of Delivering a Topical Agent to a Nasogastric Passage

As stated above, the present invention is directed to a method of delivering a topical agent, such as an anesthetic compound, an anti-inflammatory compound, a decongestant compound, or combinations thereof, to a nasogastric passage of a patient. By utilizing the method disclosed herein, it may be possible to alleviate or eliminate, for a finite amount of time, the pain and discomfort that a patient might experience when the patient is intubated with a nasogastric tube. Specifically, it may be possible to alleviate the pain and discomfort a patient might experience in the areas such as the nasal, pharyngeal, and/or esophageal passage or areas of the patient.

Accordingly, the present invention, in general, can be described as comprising the steps of introducing a nasogastric tube into the nasogastric passage and delivering a therapeutic amount of the topical local anesthetic agent to a target area in the patient. In certain embodiments, the target area can comprise the nasal, pharyngeal, and/or esophageal areas of the patient. Since the actual method of introducing a nasogastric tube into a patient is well known in the medical field, a detailed description of the procedure is not necessary. It should be noted, however, that the NG insertion procedure described herein is not to be considered tracheal intubation nor should the nasogastric tube disclosed herein be considered an endotracheal tube. The two methods for introducing an intubation tube into a patient are distinct and involve different areas of the body. As stated above, NG insertion typically involves introducing a nasogastric tube into the stomach cavity of a patient via the patient's nose and throat.

In certain embodiments, the nasogastric tube that might be used with the present invention comprises an inner tube that is disposed within an outer tube that has a larger diameter than that of the inner plastic tube. Each of the tubes can be manufactured from a plastic or an elastic material, such as polyvinyl chloride or silicone, which is known in the art. In certain embodiments, the plastic can be silver infused plastic, which can exhibit anti-microbial properties. The larger diameter of the outer plastic tube allows for the formation of a passageway between the interior surface of the outer plastic tube and the exterior surface of the small diameter inner plastic tube. The passageway between the outer and inner plastic tubes can then be used to deliver the topical local anesthetic to a target region in the patient when the nasogastric tube is properly placed. Because the nasogastric tube comprises two distinct plastic tubes, each tube can be connected to a variety of apparatuses. For example, the outer tube can be connected to an apparatus, such as a syringe, which can dispense the topical agent into the passageway between the outer and inner plastic tube while the inner tube can be connected to an apparatus for performing nasogastric aspiration. This can be accomplished by connecting conducting means, such as additional plastic tubes or tube made from an elastic material (e.g., a tube that is similar to “pigtails” or a “cuff inflation tube” used on an endotracheal tube), to each of the outer plastic tube and the inner plastic tube of the nasogastric tube. In certain embodiments, the point where the outer and inner tube connect can be generally described as having a “Y” shape wherein the inner plastic tube enters directly into the outer plastic tube thereby forming the aforementioned nasogastric tube while allowing each tube to be connected to a different apparatus. While it has been described that outer and inner tubes are connected to a different apparatus, in some embodiments, both tubes can be connected to the same apparatus.

In order to allow for the delivery of the topical agent, a portion of the nasogastric tube invention can be perforated so that when the perforated portion of the nasogastric tube is disposed adjacent to the target area or target areas of the nasogastric passage (e.g., nasal cavity, nasal-pharynx passageway, esophageal passageway, or combinations thereof), a therapeutic amount of the topical agent can be administered to the patient. This is accomplished by introducing the topical agent into the passageway between the two plastic tubes. As the topical agent flows through the passageway, it will be released from the nasogastric tube through the perforations in the outer plastic tube. In some embodiments, the perforations extend around the entire exterior surface of the relevant portion of the outer plastic tube. In other embodiments, however, the perforations extend around only a portion of the exterior surface of the outer plastic tube. Additionally, the perforations can be clustered in any type of geometric pattern (e.g., diamond shape, square shape) and the perforations themselves can have any shape (e.g, circle, square, rectangular, oval, semi-circle, quarter circle, triangular, trapezoid).

In some embodiments, the nasogastric tube comprises multiple perforated areas along the length of the tube. In other words, the nasogastric tube can comprise multiple areas, each of which comprise the aforementioned perforated portions, at different locations along the length of the nasogastric tube. In these embodiments, the nasogastric tube would allow a health care provider to target multiple target areas (nasal, pharyngeal, and/or esophageal passage) in the patient either simultaneously or at different times. For example, after the nasogastric tube is position such that a perforated portion is disposed within the patient's pharynx while another perforated portion is disposed within the nasal cavity. If the patient is experiencing discomfort in the pharynx, then the health care provider will be able to dispense the appropriate topical agent only to the perforated portion disposed within the pharynx using the conducting means connected thereto while not dispensing the topical agent to the nasal cavity region. If the patient then experiences discomfort in the nasal cavity region, the health care provider can then dispense the topical agent to the conducting means connected to the perforated portion that is disposed within the nasal cavity while discontinuing the administration of the topical agent to the pharynx. In some embodiments, the same type of topical agent can be used to treat different target areas of the patient's nasogastric passageway. In other embodiments, different topical agents are used to treat the different target areas of the patient's nasogastric passageway.

In these embodiments, a block can extend along the length of the nasogastric tube thereby separating the nasogastric tube into two distinct halves. The block, which can be made from a plastic or an elastic material, can extend from the interior surface of the outer plastic tube to the outer surface of the inner plastic tube. More specifically, the block can extend outwardly from the central axis of the nasogastric tube on a plane that is substantially parallel to the central axis. Each half can then comprise a perforated portion and each half can be connected to a different conducting means. Accordingly, the perforated portion of one half can be disposed within one target area while the perforated portion of the other half can be disposed within another target area thereby allowing the health care provider to target multiple target areas in the patient. As described above, a topical agent can then be dispensed through one half of the nasogastric tube, thereby alleviating the patient's discomfort in one target area, while also allowing the health care provider to dispense a topical agent to the other half of the nasogastric tube, when needed, thereby allowing the health care provider to treat the other target area in the patient.

In certain embodiments, the number of distinct target areas that can be treated with nasogastric tube will be dependent upon the number of distinct channels that can be made by use of a block that extends along the length of the tube. For instance, if four distinct target areas are to be treated, then two blocks can be used to separate the nasogastric tube into four distinct quarters. Each quarter can comprise a perforated portion and each quarter can be connected to a different conducting means.

In certain embodiments, the topical agent is prevented from traveling down the entire length of the nasogastric tube by a block, which can be made of a plastic or an elastic material, that is connected to and extends from the interior surface of the outer plastic tube to the outer surface of the inner plastic tube. This block, which can be placed anywhere along the length of the nasogastric tube, acts as a barrier to ensure that the topical agent does travel along the entire length of the nasogastric tube thereby preventing it from entering the portion of the nasogastric tube disposed in portions of the gastrointestinal tract such as the stomach cavity. Unlike the block described above, in general, this block can be described as having a substantially washer like shape wherein the inner plastic tube extends through the hole of the washer while the perimeter of the washer is connected to the interior surface of the outer plastic tube. That is, the block can extend outwardly from the central axis of the nasogastric tube on a plane that is substantially perpendicular to the central axis. It is noted, however, that the block should be positioned downstream from the perforations (i.e., closer to the distal end of the nasogastric tube) so that they topical agent can be dispsensed through the perforations in the outer plastic tube.

As stated above, portions of the outer plastic tube will comprise perforations. In certain embodiments, the method of the present invention calls for the health care provider to insert the nasogastric tube in the patient such that the perforated portion or portions of the nasogastric tube is disposed within the target area. In order to assist the health care provider, the outer plastic tube can be marked in the same manner that current nasogastric tubes are marked, such as with a depth markings from the distal end, so that the health care provider can insure that the tube is inserted into the patient in the proper manner while ensuring that the perforated portion of the nasogastric tube is positioned correctly. In other embodiments, the perforated portion of the nasogastric tube might be doped with a radioactive material, such as a radioactive isotope, which can be visually observed using a viewing apparatus (e.g., MRI) thereby allowing the healthcare provider to properly position the perforated portion of the tube in the target area. It should be noted, however, that other means, such as x-rays, may be used to ensure that the perforated portion of the nasogastric tube is positioned correctly in the patient.

As indicated above, when the perforated portion of the nasogastric tube is disposed within the target area, another portion of the nasogastric tube can be disposed within the gastrointestinal tract, such as within the stomach cavity.

Once the nasogastric tube is properly positioned in a manner such that the perforated portion of the tube is disposed within the target area, such as the nasal cavity and/or the nasal-pharynx passageway, the nasogastric tube can then be used to deliver the topical agent to the target area. When the topical agent is administered, the topical agent will travel through the passageway between the outer and inner plastic tubes and then exit the nasogastric tube through the perforation in the outer plastic tube thereby coming into direct contact with the target area in the patient. Since it would be well within the grasp of one skilled in the art to determine the proper amount and type of the topical agent to be used as well as the frequency in which it is to be administered, a detailed description of such will not be provided. However, in general, a sufficient amount of the topical agent should be delivered or administered to the patient such that it alleviates or reduces the pain and discomfort that the patient might be experiencing. For example, a local topical anesthetic compound can be used to numb the target area of the patient while the patient is awake thereby alleviating the patient's discomfort.

In other embodiments, a nasogastric tube that is coated, in certain portions, with the topical agent that might be used. In this embodiment, the topical agent is released into the target area over a period of time. That is, the nasogastric tube or a portion thereof can be coated with a time-release topical agent such that the topical agent is introduced or delivered to the patient over an extended period of time. While the entire nasogastric tube may be coated with the topical agent, in certain embodiments, only a portion of the nasogastric tube is coated with the anesthetic agent.

In embodiments of the invention that utilize a nasogastric tube comprising the aforementioned time-release topical local anesthetic, the method is similar to what is described in the paragraphs above. However, instead of positioning the perforations of the nasogastric tube adjacent the target area in the nasal passage, the method would call for positioning the portion of the nasogastric tube that is coated with the time-release anesthetic adjacent to the target area thereby allowing a therapeutic amount of the anesthetic to be introduced to the patient over a given amount of time.

While a variety of liquid and/or gel topical agents can be used in the present invention, in certain embodiments, an anesthetic compound, an anti-inflammatory compound, a decongestant compound, or combinations thereof are used as the topical agent. Suitable anesthetic compounds that can be used with the present invention include, without limitation, lidocaine (e.g., xylocaine), tetracaine, bupivacaine (e.g., sensorcaine or marcaine), cocaine, prilocaine, ropivacaine (e.g., NAROPIN available from APP Pharmaceuticals, LLC), articaine (e.g., SEPTOCAINE available from Septodont), dibucaine, mepivicaine (e.g., carbocaine, etidocaine, benzocaine, or combinations thereof. Suitable anti-inflammatory compounds that can be used with the present invention include, without limitation, fluticasone (e.g., FLONASE available from GlaxoSmithKline). Suitable decongestant compounds that can be used with the present invention include, without limitation, phenylephrine (e.g., NEO-SYNEPHRINE available from Bayer).

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

What is claimed is:

1. A method for delivering a topical agent to a nasogastric passage comprising:
introducing a nasogastric tube into the nasogastric passage, the nasogastric tube comprising means for delivering the topical agent to the nasal passage;
positioning the nasogastric tube such that the means for delivering the topical agent is positioned adjacent to a target area in the nasogastric passage; and
delivering the topical agent to the target area of the nasogastric passage.

2. The method according to claim 1 wherein a portion of the nasogastric tube comprises means for delivering the topical local anesthetic agent.

3. The method according to claim 1, wherein the target area comprises the nasal-pharynx passageway.

4. The method according to claim 1, wherein the target area comprises the nasal-pharynx passageway, the nasal cavity, or combinations thereof.

5. The method according to claim 1, wherein the topical agent comprises an anesthetic compound, an anti-inflammatory compound, a decongestant compound, or combinations thereof.

6. The method according to claim 5, wherein the anesthetic compound comprises lidocaine. xylocaine, or combinations thereof.

7. The method according to claim 5, wherein the anti-inflammatory compound comprises fluticasone.

8. The method according to claim 5, wherein the decongestant compound comprises phenylephrine.

9. The method according to claim 1, wherein a portion of the nasogastric tube is disposed within the gastrointestinal tract.

10. The method according to claim 9, wherein the portion of the nasogastric tube is disposed within the stomach cavity.

11. The method according to claim 1, wherein the nasogastric tube comprises an inner tube that is disposed within an outer tube, the outer tube having a diameter that is greater than the inner tube, and wherein the means for delivering the topical agent comprises perforations in a portion of the outer tube.

12. The method according to claim 1, wherein the outer tube of the nasogastric tube is connected to an apparatus for dispensing the topical agent.

13. The method according to claim 1, wherein the means for delivering the topical local anesthetic comprises a time-release topical agent.

14. A method for delivering a topical agent to a nasogastric passage comprising:
introducing a nasogastric tube into the nasogastric passage wherein the nasogastric tube comprises an inner tube that is disposed within an outer tube, the outer tube having a diameter that is greater than the inner tube, and wherein the outer tube of the nasogastric tube further comprises a perforated portion;
positioning the perforated portion of the nasogastric tube adjacent a target area in the nasogastric passage; and
delivering a therapeutic amount of said topical agent to the target area of the nasogastric passage.

15. The method according to claim 14, wherein the topical agent comprises an anesthetic compound, an anti-inflammatory compound, a decongestant compound, or combinations thereof.

16. The method according to claim 14, wherein a portion of the nasogastric tube is disposed within the gastrointestinal tract.

17. The method according to claim 16, wherein the portion of the nasogastric tube is disposed within the stomach cavity.

18. The method according to claim 14, wherein the target area comprises the nasal-pharynx passageway, the nasal cavity, or combinations thereof.

19. The method according to claim 14, wherein the topical agent comprises lidocaine. xylocaine, or combinations thereof.

20. The method according to claim 14, wherein the topical agent comprises fluticasone, phenylephrine, or combinations thereof.
